(19) United States
(12) Patent Application Publication
(10) Pub. No.: US 2023/0166014 A1
(43) Pub. Date: Jun. 1, 2023

(54) LIGHT ACCESSORY DEVICE FOR A BREAST PUMP OR THE LIKE

(71) Applicant: Lactalite, LLC, Chapin, SC (US)

(72) Inventor: Patrick Martin O’MALLEY, Chapin, SC (US)

(21) Appl. No.: 17/994,491

(22) Filed: Nov. 28, 2022

Related U.S. Application Data
(60) Provisional application No. 63/283,797, filed on Nov. 29, 2021.

(51) Int. Cl. A61M 1/06

(52) U.S. Cl. CPC A61M 1/062; A61M 2205/587

(57) ABSTRACT

A light accessory device couplable to a neck of a breast shield assembly for a breast pump, the light accessory device including: a housing; a clip structure coupled to and extensible from the housing and adapted to selectively couple the housing to the neck of the breast shield assembly; and a light source disposed within the housing and positionable adjacent to the neck of the breast shield assembly such that the light source selectively directs light towards and into the neck of the breast shield assembly or towards a breast shield of the breast shield assembly or another component of the breast pump. In addition or alternatively, the clip structure is pivotably coupled to the housing.

### CROSS-REFERENCE TO RELATED APPLICATION

The present disclosure claims the benefit of priority of co-pending U.S. Provisional Patent Application No. 63/283,797, filed on Nov. 29, 2021, and entitled “LIGHT ACCESSORY DEVICE FOR A BREAST PUMP OR THE LIKE,” the contents of which are incorporated in full by reference herein.

### TECHNICAL FIELD

The present disclosure relates generally to a light accessory device for a breast pump or the like. This light accessory device may be usefully configured to work in various other applications, beyond breast pumps.

### BACKGROUND

Breast feeding is often augmented by breast pumping, which utilizes a manual or electric pump assembly, of which there are many brands and styles. The CDC estimates over 70% of all new mothers start off breast feeding and nearly 25% are still doing so at twelve months after delivery.

Many women have difficulty knowing whether or not milk is actually being expressed from the breast while pumping. This can be due to a variety of reasons: poor lighting in the room, nighttime pumping, condensation in the breast shield, milk spray accumulating on the sides of the breast shield, and even the non-transparency of the plastic used in the breast shield.

As a result, women are often left to wonder if they are expressing milk while using a breast pump. In some cases, they must use their free hand to hold a light up to the pump for illumination. This hand is then not available to manually massage the breast, which is often necessary as milk flow slows down, or hold the baby.

Accordingly, there is a need for a light accessory device for a breast pump to allow for the illumination of the associated neck and breast shield. This device allows women to reduce the anxiety that may come with the process of breastfeeding and may also free up a woman's hand for other purposes while pumping.

### BRIEF SUMMARY

In one illustrative embodiment, the present disclosure provides a light accessory device couplable to a neck of a breast shield assembly for a breast pump, the light accessory device including: a housing; a clip structure coupled to and extensible from the housing and adapted to selectively couple the housing to the neck of the breast shield assembly; and a light source disposed within the housing and positionable adjacent to the neck of the breast shield assembly such that the light source selectively directs light towards and into the neck of the breast shield assembly or towards a breast shield of the breast shield assembly or another component of the breast pump. The clip structure is translatably coupled to and extensible from the housing. Optionally, the clip structure is translatably coupled to a track or rail structure disposed at a back portion of the housing. Optionally, the clip structure is pivotably coupled to the housing. The clip structure is extensible from an end portion of the housing. The light source is adapted to direct light from an end portion of the housing into the neck of the breast shield assembly. The clip structure includes one of a split ring and a full ring adapted to be selectively disposed circumferentially about the neck of the breast shield assembly.

In another illustrative embodiment, the present disclosure provides a breast shield assembly for a breast pump, the breast shield assembly including: a breast shield; a neck; and a light accessory device couplable to the neck, the light accessory device including: a housing; a clip structure coupled to and extensible from the housing and adapted to selectively couple the housing to the neck; and a light source disposed within the housing and positionable adjacent to the neck such that the light source selectively directs light towards and into the neck or towards the breast shield or another component of the breast pump. The clip structure is translatably coupled to and extensible from the housing. Optionally, the clip structure is translatably coupled to a track or rail structure disposed at a back portion of the housing. Optionally, the clip structure is pivotably coupled to the housing. The clip structure is extensible from an end portion of the housing. The light source is adapted to direct light from an end portion of the housing into the neck. The clip structure includes one of a split ring and a full ring adapted to be selectively disposed circumferentially about the neck.

In a further illustrative embodiment, the present disclosure provides a method of providing a light accessory device couplable to a neck of a breast shield assembly for a breast pump, the method including: providing a housing; providing a clip structure coupled to and extensible from the housing and adapted to selectively couple the housing to the neck of the breast shield assembly; and providing a light source disposed within the housing and positionable adjacent to the neck of the breast shield assembly such that the light source selectively directs light towards and into the neck of the breast shield assembly or towards a breast shield of the breast shield assembly or another component of the breast pump. The clip structure is translatably coupled to and extensible from the housing. Optionally, the clip structure is translatably coupled to a track or rail structure disposed at a back portion of the housing. The clip structure is extensible from an end portion of the housing. The light source is adapted to direct light from an end portion of the housing into the neck of the breast shield assembly.

### BRIEF DESCRIPTION OF THE DRAWINGS

The accompanying figures, wherein like reference numerals refer to identical or functionally similar elements throughout the separate views, together with the detailed description below, are incorporated in and form part of the specification, and serve to further illustrate embodiments of concepts that include the claimed invention, and explain various principles and advantages of those embodiments.

FIG. 1 is a perspective view of one illustrative embodiment of the light accessory device of the present disclosure coupled to the neck and breast shield of an associated breast pump assembly, thereby serving to illuminate the neck and breast shield when actuated.

FIG. 2 is a partial perspective view of the light accessory device of FIG. 1, highlighting the associated light device, actuation mechanism, and charging mechanism.

FIG. 3 is a rear planar view of the light accessory device of FIGS. 1 and 2, highlighting the operation of the associated breast shield coupling mechanism.

FIG. 4 is a flowchart of one illustrative embodiment of the method for providing the light accessory device of the present disclosure.

Skilled artisans will appreciate that elements in the figures are illustrated for simplicity and clarity and have not necessarily been drawn to scale. For example, the dimensions of some of the elements in the figures may be exaggerated relative to other elements to help to improve understanding of embodiments of the present disclosure.

The apparatus, device, and method components/steps have been represented, where appropriate, by conventional symbols in the drawings, showing only those specific details that are pertinent to understanding the embodiments of the present disclosure so as not to obscure the disclosure with details that will be readily apparent to those of ordinary skill in the art having the benefit of the description herein.

### DETAILED DESCRIPTION

The light accessory device for a breast pump described herein allows for a user to better determine whether or not milk is actually being expressed from the breast when using a breast shield assembly and breast pump. The device is designed to allow for simple attachment to a wide variety of commercially available breast shield assemblies and breast pumps. With the use of this device, the user will no longer have to use their free hand to hold a light to the breast shield assembly and breast pump for illumination, which makes the free hand available to manually massage the breast—which is often a necessary task as milk flow begins to slow down.

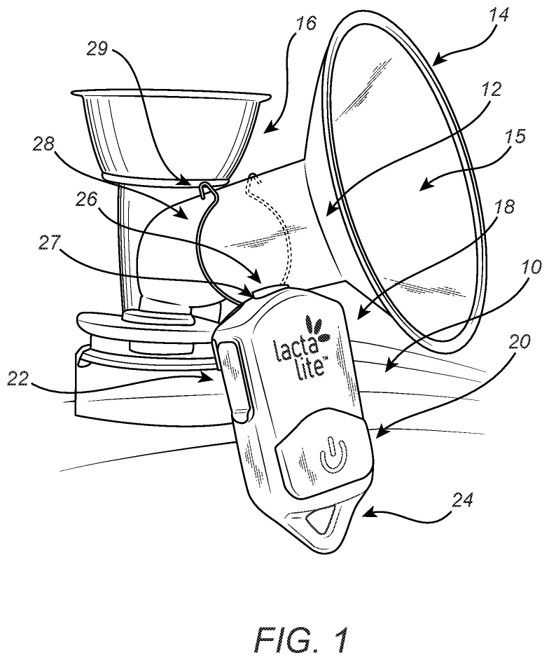

FIG. 1 is a perspective view of one illustrative embodiment of the light accessory device 10 of the present disclosure coupled to the neck 12 and breast shield 14 (collectively referred to as a breast shield assembly 15) of an associated breast pump assembly 16, thereby serving to illuminate the neck 12 and breast shield 14 when actuated. A typical breast pump assembly 16 includes the breast shield 14 that is designed to be contacted with the female breast and the neck 12 that is attached to the suction tube and other components of the breast pump assembly 16 to carry the expressed milk from the breast shield 14 to a collection bottle. Typically, the neck 12 is a substantially cylindrical structure and the breast shield 14 is a substantially conical structure, although other suitable shapes are possible. The neck 12 and breast shield 14 may be separate, coupled components, or may be integrally formed. The neck 12 and breast shield 14 are typically manufactured from a medical grade, cleanable/sterilizable plastic material or the like. These components are generally well known to those of ordinary skill in the art.

The light accessory device 10 includes a prismatic housing 18 including an actuation switch 20, a charging port 22, and an eyelet 24 to which a carrying strap, keychain or the like may be attached. The housing 18, which may be multi-piece, and eyelet 24 are manufactured from a durable plastic or other moisture-resistant material or the like, while the actuation switch 20 is manufactured from a plastic or flexible rubber or other moisture-resistant material or the like, such that the actuation switch 20 may be depressed in relation to the housing 18 while maintaining a moisture-resistant seal. Likewise, the charging port 22 may be covered with a flexible rubber or other moisture-resistant flap or plug that serves to protect the enclosed battery, circuitry, and charging connection(s). In the embodiment illustrated, the actuation switch 20 is relatively large and is located in a central or end portion of the front portion of the housing 18, such that it may be easily actuated with a thumb or finger push. In the embodiment, illustrated, the charging port 22 is located along a side portion of the housing 18, although other locations may be utilized equally.

The light accessory device 10 includes a light source 26 that is configured to be selectively disposed adjacent to and direct light into the neck 12 and breast pump shield 14 and an extensible clip structure 28 that is configured to selectively secure the housing 18 to the neck 12 with the light source 26 disposed adjacent to and directing light into the neck 12 and breast pump shield 14. In this illustrative embodiment, the light source 26 may include one or more LED light devices or the like that are configured to direct light radially into the neck 12 through a port 27 disposed through an end portion of the housing 18. The clip structure 28 may include a plastic or metallic split-ring structure that has a degree of deflectability and provides a concentric spring force, such that the clip structure 28 may securely accommodate different-circumference necks 12. Alternatively, the clip structure 28 may include a plastic or metallic full-ring structure (or flexible/elastic strap structure) that is simply disposed about the neck 12, which may have a constant or gradient diameter along a length thereof. In the split-ring configuration, the clip structure 28 may have a hook or rounded portion 29 at an end of each arm thereof, promoting ease of use and safety by eliminating sharp points.

Optionally, the light source 26 delivers light with a wavelength that accomplishes a disinfection function within the neck 12. The light source 26 may be of any color, including white, colors, or a “black light” that emits ultraviolet radiation. The light source 26 may have the option to switch between multiple colors, via the actuation switch 20. The actuation switch 20 controlling the light source 26 may be a button switch, a rocker switch, a clicker switch, or utilize any other method of starting or stopping electric current. In one illustrative embodiment, a single or quick press of the actuation switch 20 turns the light source 26 on “low,” while a second or extended press of the actuation switch 20 turns the light source 26 on “high,” with a subsequent press of the actuation switch 20 turning the light source off, for example.

The housing 18 contains internal batteries or allows for the connection of an external power pack through the charging port 22. The housing 18 may also have a removable cover or component that allows for the internal batteries to be changed, for example.

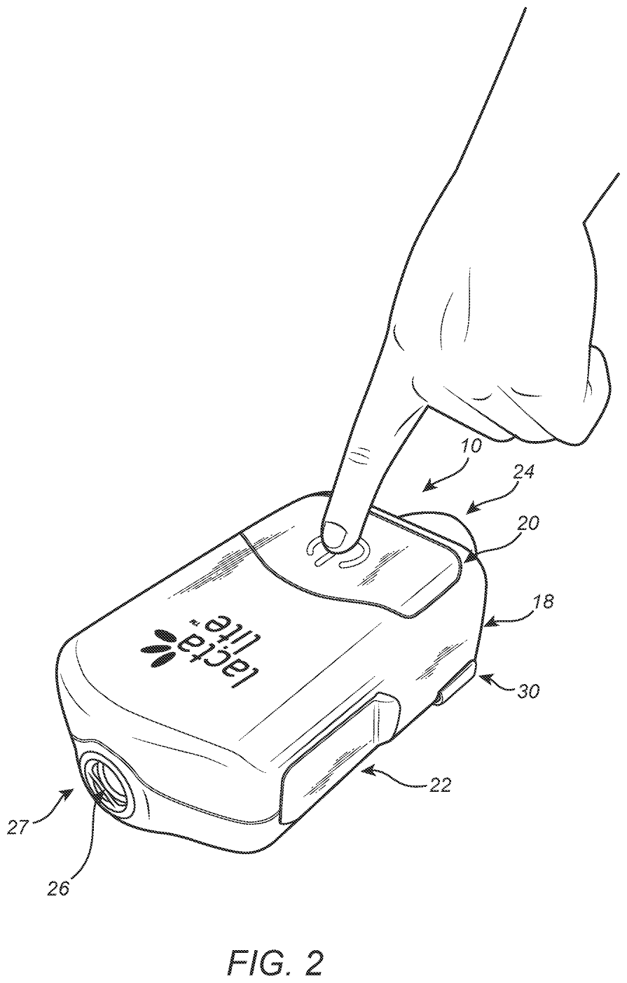

FIG. 2 is a partial perspective view of the light accessory device 10 of FIG. 1, highlighting the associated light source 26, light port 27, actuation switch 20, and charging port 22. Here, it can be seen that the clip structure 28 (FIG. 1) is coupled to a slider 30 that is configured to selectively translate along the length of the housing 18, such that the clip structure 28 may be extended from a “stowed” position adjacent to the back of the housing 18 to “use” position extended from the housing 18 (as illustrated in FIG. 1). This mechanism is described in greater detail below. The slider 30 may also be manufactured from a rigid plastic or metallic material.

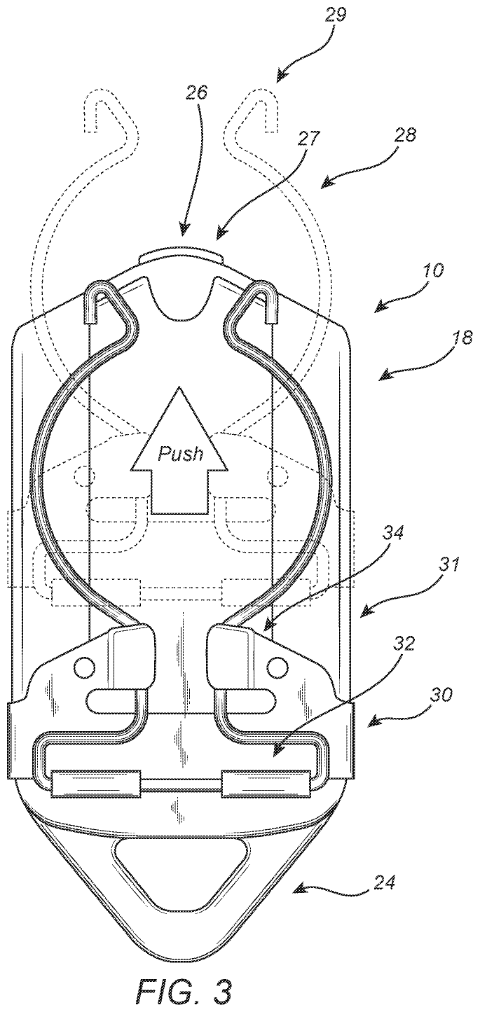

FIG. 3 is a rear planar view of the light accessory device 10 of FIGS. 1 and 2, highlighting the operation of the associated breast shield coupling mechanism. Here, again, it can be seen that the clip structure 28 is coupled to a slider 30 that is configured to selectively translate along the length of the housing 18, such that the clip structure 28 may be extended from a “stowed” position adjacent to the back of the housing 18 to “use” position extended from the housing 18. This slider 30 includes side hook arms that translatably grasp rails or grooves 31 disposed in/on the back or in/on the sides of the housing 18, creating a “track” along with the slider 30 translates with a “push” or “pull” motion by a user, optionally by the thumb of the user. Detents may be provided at full retraction and full extension of the clip structure 28, as well as at intermediate positions if desired. As illustrated, the clip structure 28 is formed from a unitary wire. A bottom portion of this unitary wire is captured by one or more pivot connections 32 associated with the slider 30, such that the loop of the clip structure 28 may be pivoted away from and towards the back of the housing 18, thereby, allowing the angle of the housing 18 and light source 26 to be adjusted relative to the neck 12 (FIG. 1) of the breast pump assembly 16 (FIG. 1). The middle portion of the unitary wire is selectively captured by one or more retention hooks 34, thereby securing the unitary wire to the back of housing 18 in an unpivoted configuration, or releasing the housing 18 to pivot away from the back of the housing 18 with a “squeeze’ of the unitary wire. Other pivot and translation mechanisms may be used equally, provided they function in a similar manner. This pivoting functionality allows the light to be selectively directed from the end portion (or any other portion) of the housing 18 alternatively towards/into the neck 12 of the breast shield assembly 15, the breast shield 14 itself, or another component of the breast pump 16.

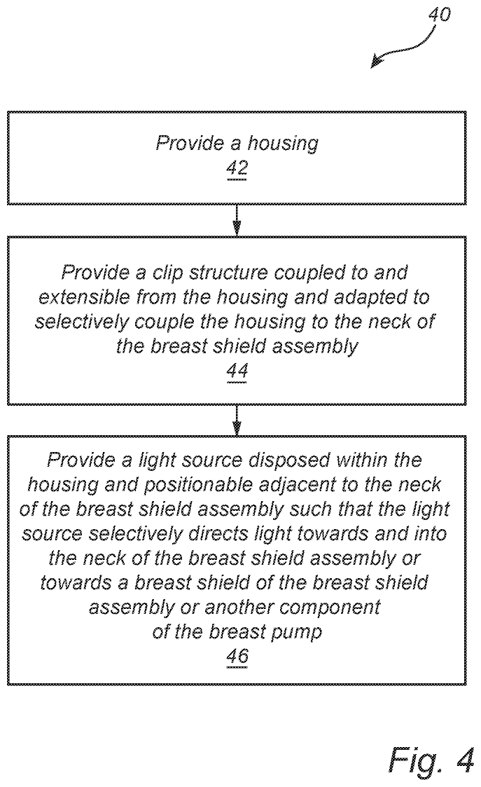

Referring to FIG. 4, in a further illustrative embodiment, the present disclosure provides a method 40 of providing a light accessory device couplable to a neck of a breast shield assembly for a breast pump, the method including: providing a housing 42; providing a clip structure coupled to and extensible from the housing and adapted to selectively couple the housing to the neck of the breast shield assembly 44; and providing a light source disposed within the housing and positionable adjacent to the neck of the breast shield assembly such that the light source selectively directs light towards and into the neck of the breast shield assembly or towards a breast shield of the breast shield assembly or another component of the breast pump 46. The clip structure is translatably coupled to and extensible from the housing. Optionally, the clip structure is translatably coupled to a track or rail structure disposed at a back portion of the housing. The clip structure is extensible from an end portion of the housing. The light source is adapted to direct light from an end portion of the housing into the neck of the breast shield assembly.

Thus, again, the light accessory device for a breast pump described herein allows for a user to better determine whether or not milk is actually being expressed from the breast when using a breast shield assembly and breast pump. The device is designed to allow for simple attachment to a wide variety of commercially available breast shield assemblies and breast pumps. With the use of this device, the user will no longer have to use their free hand to hold a light to the breast shield assembly and breast pump for illumination, which makes the free hand available to manually massage the breast—which is often a necessary task as milk flow begins to slow down.

One of ordinary skill in the art appreciates that various modifications and changes can be made without departing from the scope of the invention as set forth in the claims below. Accordingly, the specification and figures are to be regarded in an illustrative rather than a restrictive sense, and all such modifications are intended to be included within the scope of present teachings.

The benefits, advantages, solutions to problems, and any element(s) that may cause any benefit, advantage, or solution to occur or become more pronounced are not to be construed as a critical, required, or essential features or elements of any or all the claims. The invention is defined solely by the appended claims including any amendments made during the pendency of this application and all equivalents of those claims as issued.

Moreover in this document, relational terms such as first and second, top and bottom, and the like may be used solely to distinguish one entity or action from another entity or action without necessarily requiring or implying any actual such relationship or order between such entities or actions. The terms “comprises,” “comprising,” “has,” “having,” “includes,” “including,” “contains,” “containing,” or any other variation thereof, are intended to cover a non-exclusive inclusion, such that a process, method, article, or apparatus that comprises, has, includes, contains a list of elements does not include only those elements but may include other elements not expressly listed or inherent to such process, method, article, or apparatus. An element proceeded by “comprises a,” “has a,” “includes a,” “contains a,” does not, without more constraints, preclude the existence of additional identical elements in the process, method, article, or apparatus that comprises, has, includes, contains the element. The terms “a” and “an” are defined as one or more unless explicitly stated otherwise herein. The terms “substantially,” “essentially,” “approximately,” “about,” or any other version thereof, are defined as being close to as understood by one of ordinary skill in the art. The term “coupled” as used herein is defined as connected, although not necessarily directly and not necessarily mechanically. A device or structure that is “configured” in a certain way is configured in at least that way, but may also be configured in ways that are not listed.

The Abstract is provided to allow the reader to quickly ascertain the nature of the technical disclosure. It is submitted with the understanding that it will not be used to interpret or limit the scope or meaning of the claims. In addition, in the foregoing Detailed Description, it can be seen that various features are grouped together in various embodiments for the purpose of streamlining the disclosure. This method of disclosure is not to be interpreted as reflecting an intention that the claimed embodiments require more features than are expressly recited in each claim. Rather, as the following claims reflect, inventive subject matter lies in less than all features of a single disclosed embodiment. Thus, the following claims are hereby incorporated into the Detailed Description, with each claim standing on its own as a separately claimed subject matter.

What is claimed is:

1. A light accessory device couplable to a neck of a breast shield assembly for a breast pump, the light accessory device comprising:
a housing;
a clip structure coupled to and extensible from the housing and adapted to selectively couple the housing to the neck of the breast shield assembly; and
a light source disposed within the housing and positionable adjacent to the neck of the breast shield assembly such that the light source selectively directs light towards and into the neck of the breast shield assembly or towards a breast shield of the breast shield assembly or another component of the breast pump.

2. The light accessory device of claim 1, wherein the clip structure is translatably coupled to and extensible from the housing.

3. The light accessory device of claim 2, wherein the clip structure is translatably coupled to a track or rail structure disposed at a back portion of the housing.

5. The light accessory device of claim 1, wherein the clip structure is pivotably coupled to the housing.

6. The light accessory device of claim 1, wherein the clip structure is extensible from an end portion of the housing.

7. The light accessory device of claim 1, wherein the light source is adapted to direct light from an end portion of the housing into the neck of the breast shield assembly.

8. The light accessory device of claim 1, wherein the clip structure comprises one of a split ring and a full ring adapted to be selectively disposed circumferentially about the neck of the breast shield assembly.

9. A breast shield assembly for a breast pump, the breast shield assembly comprising:
a breast shield;
a neck; and
a light accessory device couplable to the neck, the light accessory device comprising:
a housing;
a clip structure coupled to and extensible from the housing and adapted to selectively couple the housing to the neck; and
a light source disposed within the housing and positionable adjacent to the neck such that the light source selectively directs light towards and into the neck or towards the breast shield or another component of the breast pump.

10. The breast shield assembly of claim 9, wherein the clip structure is translatably coupled to and extensible from the housing.

11. The breast shield assembly of claim 10, wherein the clip structure is translatably coupled to a track or rail structure disposed at a back portion of the housing.

12. The breast shield assembly of claim 9, wherein the clip structure is pivotably coupled to the housing.

13. The breast shield assembly of claim 9, wherein the clip structure is extensible from an end portion of the housing.

14. The breast shield assembly of claim 9, wherein the light source is adapted to direct light from an end portion of the housing into the neck.

15. The light accessory device of claim 1, wherein the clip structure comprises one of a split ring and a full ring adapted to be selectively disposed circumferentially about the neck.

16. A method of providing a light accessory device couplable to a neck of a breast shield assembly for a breast pump, the method comprising:
providing a housing;
providing a clip structure coupled to and extensible from the housing and adapted to selectively couple the housing to the neck of the breast shield assembly; and
providing a light source disposed within the housing and positionable adjacent to the neck of the breast shield assembly such that the light source selectively directs light towards and into the neck of the breast shield assembly or towards a breast shield of the breast shield assembly or another component of the breast pump.

17. The method of claim 16, wherein the clip structure is translatably coupled to and extensible from the housing.

18. The method of claim 17, wherein the clip structure is translatably coupled to a track or rail structure disposed at a back portion of the housing.

19. The method of claim 16, wherein the clip structure is extensible from an end portion of the housing.

20. The method of claim 16, wherein the light source is adapted to direct light from an end portion of the housing into the neck of the breast shield assembly.
